(19) United States
(12) Patent Application Publication
(10) Pub. No.: US 2023/0233251 A1
(43) Pub. Date: Jul. 27, 2023

(54) SYMPATHETIC NERVOUS SYSTEM RESPONSE TO BLOOD FLOW ALTERATIONS IN RENAL VASCULATURE FOR PATIENT STRATIFICATION IN RENAL DENERVATION

(71) Applicant: KONINKLIJKE PHILIPS N.V., EINDHOVEN (NL)

(72) Inventors: James David CEZO, SAN DIEGO, CA (US); Becky INDERBITZEN, SAN DIEGO, CA (US)

(21) Appl. No.: 18/098,943

(22) Filed: Jan. 19, 2023

Related U.S. Application Data
(60) Provisional application No. 63/302,445, filed on Jan. 24, 2022.

(51) Int. Cl. A61B 18/14

(52) U.S. Cl. CPC A61B 18/1492; A61B 2018/00404

(57) ABSTRACT

A system is provided including an intravascular catheter or guidewire and a processor circuit. The processor circuit determines a metric corresponding to the state of the sympathetic nervous system. The processor circuit then controls the intravascular catheter to alter the blood flow within the vessel. The processor circuit then determines another metric corresponding to the state of the sympathetic nervous system while the blood flow is altered. The processor circuit then provides an output based on the metrics obtained while the blood flow was not altered and while the blood flow was altered.

### CROSS-REFERENCE TO RELATED APPLICATIONS

This application claims priority to and the benefit of U.S. Provisional Application No. 63/302,445, filed Jan. 24, 2022, which is incorporated by reference herein in its entirety.

### TECHNICAL FIELD

The present disclosure relates generally to renal denervation. In particular, a patient’s sympathetic nervous system is monitored during stimulation of the renal nerves by altering blood flow within the renal arteries to stratify patients based on their likelihood to respond to a renal denervation procedure.

### BACKGROUND

Physicians use many different medical diagnostic systems and tools to monitor a patient’s health and diagnose medical conditions. In the field of assessing and treating hypertension in patients, various systems and devices are used to monitor a patient’s condition and perform treatment procedures. One treatment procedure used to address hypertension of a patient is renal denervation. Renal denervation involves ablating or otherwise disabling the nerves of the renal artery. Because the renal nerves cause the renal artery to expand or contract in response to various stimuli, the renal nerves may be a cause of unnecessary high blood pressure in a patient. By disabling these nerves, blood pressure may be decreased.

However, renal denervation is not an effective treatment in all patients or at all locations within the renal vasculature of a patient. It is often difficult for a physician to determine whether a renal denervation will effectively address hypertension for a patient as results of renal denervation are highly patient-specific. As a result, a physician may perform a renal denervation procedure without success. This may be because the patient was not a patient which would respond positively to a renal denervation procedure or because the renal denervation procedure was performed in an incorrect region of the renal vasculature. Performing a renal denervation procedure with little to no effect on the patient unnecessarily subjects a patient to a traumatic and time-consuming procedure and wastes costly resources.

### SUMMARY

Embodiments of the present disclosure are systems, devices, and methods for stratifying patients for renal denervation based on monitoring sympathetic nervous response to blood flow alteration in the renal arteries. Aspects of the disclosure advantageously assist physicians in determining whether a patient would be an appropriate candidate for a renal denervation procedure and whether a renal denervation procedure performed previously was effective.

In some aspects, an endovascular device may be positioned within a renal artery of a patient. The endovascular device adjusts blood flow within the renal arty and measures the sympathetic response to the blood flow alteration. To adjust blood flow, the endovascular device may include a balloon or a pump. A balloon of the endovascular device is positioned within a rental artery and is expanded to restrict blood flow through the renal artery. A pressure sensor and/or flow sensor is positioned distal of the balloon to measure changes in blood flow as the balloon expands. A pump of the endovascular device may include an inlet to be positioned within the renal artery leading to an outlet to be positioned within the aorta. The pump moves blood from the renal artery to the aorta to reduce blood flow within the renal artery.

The endovascular device includes a pressure sensor for monitoring blood pressure while blood flow is altered within the renal artery. If a renal denervation procedure has not been performed and if the blood pressure of the patient changes to the extent of satisfying a threshold, a processor circuit of the system determines that the patient is a good candidate for renal denervation. If a renal denervation procedure was already performed, the processor circuit may determine that it was not successful and recommend additional treatment. If a renal denervation procedure has not been performed and if the blood pressure of the patient does not change to the extent of satisfying a threshold, the processor circuit determines that the patient is not a good candidate for renal denervation. If a renal denervation procedure was already performed, the processor circuit may determine that it was successful.

In an exemplary aspect, a system is provided. The system includes an intravascular catheter or guidewire sized and shaped for positioning within a first blood vessel of a patient; and a processor circuit configured for communication with the intravascular catheter or guidewire, wherein the processor circuit is configured to: determine, using the intravascular catheter or guidewire, a first metric corresponding to a first state of a sympathetic nervous system of the patient; control the intravascular catheter or guidewire to alter a blood flow within the first blood vessel; determine, using the intravascular catheter or guidewire, a second metric corresponding to a second state of the sympathetic nervous system of the patient, the second state of the sympathetic nervous system resulting from the altered blood flow within the first blood vessel; and provide, to a display in communication with the processor circuit, an output based on the first metric and the second metric.

In one aspect, the intravascular catheter or guidewire comprises a blood flow sensor, and the processor circuit is configured to receive, from the blood flow sensor, blood flow data representative of the blood flow within the first blood vessel. In one aspect, the processor circuit is configured to control the intravascular catheter or guidewire to alter the blood flow based on the blood flow data. In one aspect, the intravascular catheter or guidewire comprises a balloon, and to control the intravascular catheter or guidewire to alter the blood flow, the processor circuit is configured to control expansion of the balloon within the first blood vessel to restrict the blood flow. In one aspect, the intravascular catheter or guidewire comprises a pump, and to control the intravascular catheter or guidewire to alter the blood flow, the processor circuit is configured to control the pump to: move blood from the first blood vessel to a second blood vessel; or move blood from the second blood vessel to the first blood vessel. In one aspect, the intravascular catheter or guidewire comprises a pressure sensor, and the first metric comprises a first blood pressure metric and the second metric comprises a second blood pressure metric. In one aspect, the intravascular catheter or guidewire comprises at least one pressure sensor and at least one flow sensor, and the first metric comprises a first fluid resistance metric and the second metric comprises a second fluid resistance metric. In one aspect, the intravascular catheter or guidewire comprises an electrode, and the first metric corresponds to a first voltage metric and the second metric corresponds to a second voltage metric. In one aspect, the intravascular catheter or guidewire comprises a strain sensor, and the first metric corresponds to a first resistance metric and the second metric corresponds to a second resistance metric. In one aspect, the processor circuit is configured to perform a comparison based on the first metric and the second metric. In one aspect, the comparison comprises a determination of whether a difference between the first metric and the second metric exceeds a threshold difference. In one aspect, the blood vessel is a renal artery, the comparison comprises a determination of whether a renal denervation is recommended for the patient, and the output comprises a visual representation of the determination. In one aspect, the blood vessel is a renal artery, the comparison comprises a determination of whether a renal denervation was successful, and the output comprises a visual representation of the determination.

In an exemplary aspect, a method is provided. The method includes determining, with a processor circuit in communication with an intravascular catheter or guidewire, a first metric corresponding to a first state of a sympathetic nervous system of the patient using an intravascular catheter or guidewire positioned within a blood vessel; controlling, with the processor circuit, the intravascular catheter or guidewire to alter a blood flow within the blood vessel using the intravascular catheter or guidewire; determining, with the processor circuit, a second metric corresponding to a second state of the sympathetic nervous system of the patient using the intravascular catheter or guidewire, the second state of the sympathetic nervous system resulting from the altered blood flow within the blood vessel; and providing, with the processor circuit, an output based on the first metric and the second metric to a display in communication with the processor circuit.

In an exemplary aspect, a system is provided. The system includes an intravascular catheter or guidewire sized and shaped to be positioned within a renal artery of a patient, wherein the intravascular catheter guidewire comprises: one or more sensors; and at least one of a balloon or a pump; and a processor circuit configured for communication with the intravascular catheter or guidewire, wherein the processor circuit is configured to: determine, using the one or more sensors, a first metric corresponding to a first state of a sympathetic nervous system of the patient; control at least one of the balloon or the pump to alter a blood flow within renal artery, thereby changing the sympathetic nervous system from the first state to a second state; determine, using the one or more sensors, a second metric corresponding to the second state of the sympathetic nervous system; and provide, to a display in communication with the processor circuit, an output based on the first metric and the second metric.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a flow diagram of a method of automatically segmenting a vessel and generating a treatment plan for the vessel based on coregistration of physiology data and extraluminal data, according to aspects of the present disclosure.

FIG. 2 is a schematic diagram of a data acquisition and blood flow alteration system, according to aspects of the present disclosure.

FIG. 3 is a schematic diagram of an intravascular device disposed within the human renal anatomy, according to aspects of the present disclosure.

FIG. 4 is a schematic diagram of an endovascular device, according to aspects of the present disclosure.

FIG. 5 is a diagrammatic view of hemodynamic data associated with a blood flow alteration procedure, according to aspects of the present disclosure.

FIG. 6 is a schematic diagram of an endovascular device, according to aspects of the present disclosure.

FIG. 7 is a schematic diagram of an endovascular device, according to aspects of the present disclosure.

FIG. 8 is a schematic diagram of an endovascular device, according to aspects of the present disclosure.

FIG. 9 is a schematic diagram of an endovascular device, according to aspects of the present disclosure.

FIG. 10 is a schematic diagram of an endovascular device, according to aspects of the present disclosure.

FIG. 11 is a schematic diagram of an endovascular device, according to aspects of the present disclosure.

FIG. 12 is a schematic diagram of an endovascular device, according to aspects of the present disclosure.

FIG. 13 is a schematic diagram of a processor circuit, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

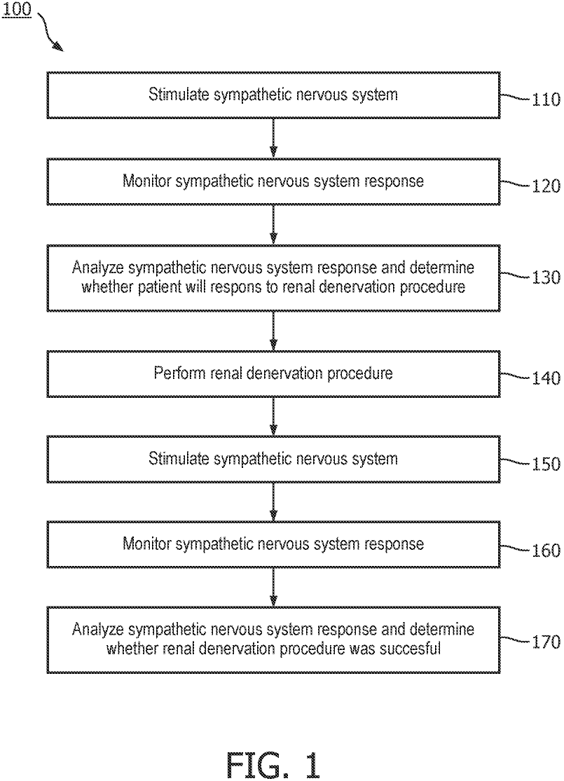

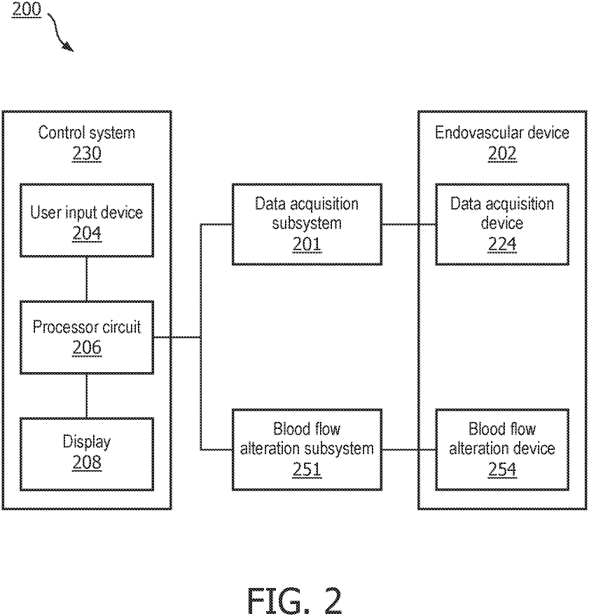

FIG. 1 is a flow diagram of a method 100 of automatically segmenting a vessel and generating a treatment plan for the vessel based on coregistration of physiology data and extraluminal data, according to aspects of the present disclosure. The method 100 may describe an automatic segmentation of a vessel to detect segments of interest using co-registration of invasive physiology and x-ray images. As illustrated, the method 100 includes a number of enumerated steps, but embodiments of the method 100 may include additional steps before, after, or in between the enumerated steps. In some embodiments, one or more of the enumerated steps may be omitted, performed in a different order, or performed concurrently. The steps of the method 100 can be carried out by any suitable component within a diagnostic system and all steps need not be carried out by the same component. In some embodiments, one or more steps of the method 100 can be performed by, or at the direction of, a processor circuit of the diagnostic system 100, including, e.g., the processor 260 (FIG. 2) or any other component.

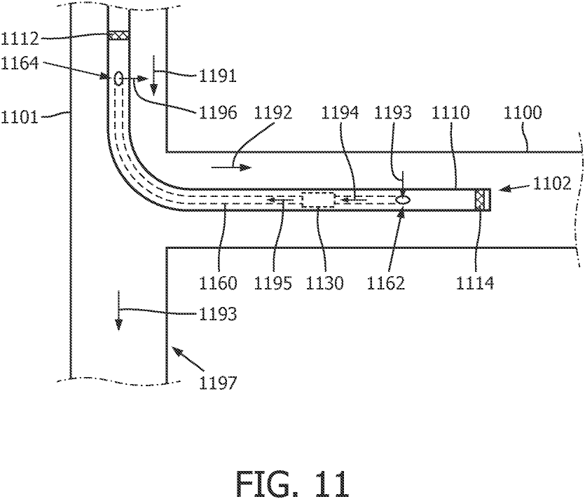

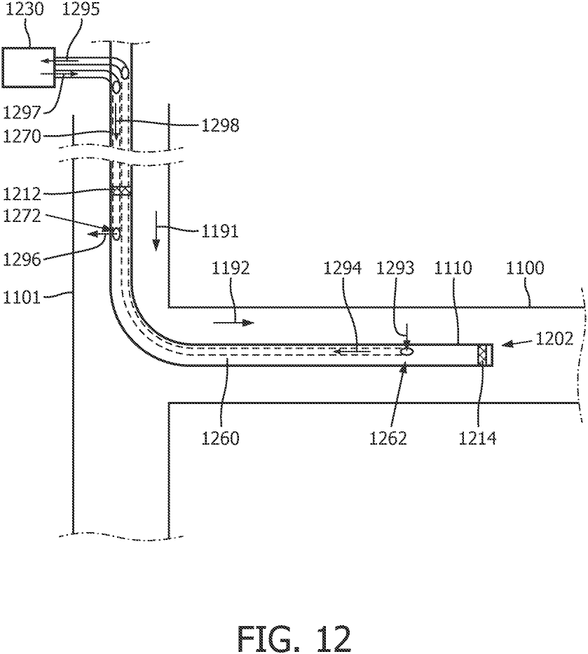

At step 110, the method 100 includes stimulating the sympathetic nervous system. The sympathetic nervous system of a patient may be stimulated in a number of ways. In one embodiment, the sympathetic nervous system may be stimulated with a blood flow modification device. The blood flow modification device may include an endovascular device, such as intravascular catheter that is sized and shaped for positioning in a blood vessel, such as the blood vessel shown in FIG. 11 or the blood vessel shown in FIG. 12. Blood flow modifications may be used to assess sympathetic tone in the renal artery. In one example, a compliant balloon is proximal to, distal to, or between various physiological sensors (e.g., on or more pressure sensors, flow sensors, or other sensors). The compliant balloon can be dilated to restrict blood flow. Full dilation of the balloon will put the balloon surface in contact with the intimal surface of the renal artery, fully restricting blood flow. The reduction of flow to the kidney and reduction of pressure will alter the sympathetic drive from the renal nerve. This in turn will impact the patient’s blood pressure. Blood pressure changes over time will indicate the patient’s receptiveness to renal denervation therapies.

The balloon of the device may be placed in the renal artery or distal branches. This may reduce flow and lead to a pressure drop on the distal side of the balloon. This then alters sympathetic response and leads to global drop in blood pressure. The balloon may completely occlude the vessel or partially occlude it. A blood flow sensor at the tip of the intravascular device, or at any location distal of the balloon, may determine the extent of occlusion. A second distal sensor could be a pressure sensor. The pressure sensor could also be used to monitor the extent of occlusion of the vessel by the balloon. In some embodiments, a flow sensor alone may be distal of the balloon. In some embodiments, a pressure sensor alone may be distal of the balloon. In some embodiments, both a flow sensor and a pressure sensor may be distal of the balloon.

In some embodiments blood flow may be altered to stimulate the sympathetic nervous system with a blood pump. The catheter may be designed with an inline or external blood pump. This pump may draw blood from a distal end or region of the catheter which is placed into the renal artery. The pump may push blood through a proximal hole in the catheter positioned in the aorta. In that regard, the pump may reduce the blood flow and pressure within the renal artery, altering the sympathetic drive from the renal nerve. This in turn impacts the patient’s blood pressure which may be monitored to assess the likelihood of positive response to renal denervation procedures.

At step 120, the method 100 includes monitoring the sympathetic nervous system for a response to the stimulation. Monitoring the sympathetic nervous system for a response may include measuring a global blood pressure of the patient. For example, the global blood pressure may be measured by a pressure sensor on the catheter proximal to the balloon or otherwise configured to monitor the blood pressure proximal to the balloon. In some embodiments, blood pressure and/or flow may also be measured on the distal side of the balloon. The global blood pressure may also be measured with an external device, such as a blood pressure cuff. In some embodiments, global blood pressure may be measured with an arterial line pressure sensor.

In some embodiments, a flow measurement distal of the balloon or proximal to the balloon may be used to monitor the sympathetic nervous system response to stimulation. In some embodiments, a single flow sensor may be positioned distal or proximal to the balloon. In some embodiments, one or more pressure sensors and/or flow sensors may be used to measure a flow resistance or impedance across the region of the balloon, proximal to the balloon, or distal of the balloon to monitor the sympathetic response. The device described may be placed in the main renal artery or may be placed in distal branches of the renal vasculature.

In some embodiments, the balloon may include contact sensors on an outer surface of the balloon. These contact sensors (e.g., electrodes) may measure electric potential changes of the artery wall pre- and post-denervation. These contact sensors may be attached to the balloon and may monitor the renal nerves. In some embodiments, the contact sensors may replace any of the proximal or distal sensors described previously. In some embodiments, a device may include both contact sensors as well as proximal and/or distal pressure sensors and/or flow sensors.

In some embodiments, the balloon may include one or more strain sensors which can detect changes to the vascular distention or constriction of the surrounding vessel. The strain sensors may replace the electrodes measuring electrical potential or the device may include both one or more strain sensors as well as one or more electrodes. The strain sensors may replace any of the pressure or flow sensors as well or the device may include both the strain sensors as well as any of the pressure or flow sensors described herein. In some embodiments, the device may include the strain sensors, the electrodes, and any of the pressure or flow sensors described herein. The strain sensors may be used to monitor sympathetic response pre- and/or post-denervation.

At step 130, the method 100 includes analyzing the sympathetic nervous system response and determining whether the patient will respond to a renal denervation procedure. For example, a processor circuit of the system may identify to what extent the blood pressure of the patient changes while the sympathetic nervous system is under stimulation. If no change is observed, the processor circuit may determine that the patient is not a good candidate for renal denervation. However, if a change is observed, including a change satisfying a threshold, the processor circuit may determine that the patient is a good candidate for renal denervation. In other embodiments, as will be described, the processor circuit may alternatively or additionally analyze any of an electrical potential or impulse at a region of the renal vasculature, a strain from a strain sensor, blood flow, or any other parameter relating to measuring the response of the sympathetic nervous system.

At step 140, the method 100 includes performing a renal denervation procedure. A renal denervation procedure may include ablating the renal nerves proximate to the renal artery such that they are disabled. As a result, blood pressure in the may be reduced. After a renal denervation procedure, the steps 110 through 130 may be performed again to determine if the renal denervation procedure was successful.

At step 150, the method 100 includes stimulating the sympathetic nervous system. Step 150 may include any of the steps or principles described with reference to step 110 of the method 100.

At step 160, the method 100 includes monitoring the sympathetic nervous system for a response to the stimulation. Step 160 may include any of the steps or principles described with reference to step 120 of the method 100.

At step 170, the method 100 includes analyzing the sympathetic nervous system response and determining whether the renal denervation procedure was successful. Step 170 may include any of the steps or principles described with reference to step 130 of the method 100. Specifically, after a renal denervation procedure is performed, the physician expects to see a decrease in change in blood pressure (or any other parameters previously described) while the sympathetic nervous system is under stimulation. For example, if, at step 170, the processor circuit observes that there is no change or little to no change in blood pressure during stimulation, the processor circuit may determine that the renal denervation procedure was successful. If, however, the processor circuit observes a change, or change satisfying a threshold, to blood pressure during stimulation, the processor circuit may determine that the renal denervation procedure was not successful. In some aspects, the processor circuit may then further direct the user to perform an additional renal denervation procedure, as outlined in step 140. In some aspects, the processor circuit may instruct the user to navigate the device to a different location and perform an additional renal denervation procedure as outline in step 140 or may instruct the user to move the device to a different location and perform any or all of the steps of the method 100 again. Aspects of the steps of the method 100 will be described with more detail throughout the description given with reference to the following figures.

In some aspects, any of the systems, devices, sensors, methods, principles, or any teachings of the present invention may be substantially similar to the teachings of U.S. Provisional Application No. 63/300,536, filed Jan. 18, 2022, which is incorporated by reference herein in its entirety.

FIG. 2 is a schematic diagram of a data acquisition and blood flow alteration system 200, according to aspects of the present disclosure. In some embodiments, and as shown in FIG. 2, the system 200 may include a control system 230, one or more subsystems, and one or more endovascular devices, such as the endovascular device 202.

The system 200 shown in FIG. 2 may advantageously assist a physician in assessing causes of hypertension in some patients and may assist a physician in determining whether a renal denervation procedure will likely be successful for a particular patient and/or whether a renal denervation procedure already performed was successful. In addition, the system 200 shown in FIG. 2 may be configured to identify whether a patient is likely to respond positively to a renal denervation procedure. For example, the system 200 may be configured to stimulate the sympathetic nervous system of the patient and measure a response to the stimulation. In some examples, the system 200 may stimulate the sympathetic nervous system by altering the blood flow within a renal artery of a patient. By analyzing the response of the patient to the stimulation of the sympathetic nervous system, the system 200 may be able to determine, based on the physiological response of the patient to stimulation, whether a patient’s hypertension may be remedied or aided through a renal denervation procedure. The system 200 may also be able to quantify the effect of a previous renal denervation procedure on the response of the patient and therefore predict whether a previous renal denervation procedure is likely to be successful in remedying hypertension within the patient.

The control system 230 may be configured to generate various commands to control subsystems, such as the data acquisition subsystem 201 and/or the blood flow alteration subsystem 251. The control system 230 may be additionally configured to generate commands to control various devices. For example, the control system 230 may be configured to generate commands to control the endovascular device 202. In some embodiments, the control system 230 may be configured to generate command signals to control one or more devices, such as the data acquisition device 224. The data acquisition device 224 may include various sensors, such as flow sensors, flow velocity sensors, pressure sensors, electrodes, strain sensors, or any other measurement devices. In addition, the control system 230 may be configured to generate command signals to control one or more blood flow alteration devices, such as the blood flow alteration device 254 shown in FIG. 2.

The control system 230 may be any suitable device or system. For example, the control system 230 may include a user input device 204, a processor circuit 206, and/or a display 208. The control system 230 may include additional devices, components, or elements. In some embodiments, the control system 230 may be a computer, such as a laptop, a tablet device, or any other suitable computational device. In some embodiments, the control system 230 may include additional elements related to communication between the control system 230, or the processor circuit 206 of the control system 230, and other systems, subsystems, or devices. For example, the control system 230 may include an interface module. In some examples, the control system 230 may include a patient interface module (PIM).

In some embodiments, the control system 230 may additionally be configured to receive various data from other systems, subsystems, or devices. For example, the control system may be configured to receive data related to blood flow, the velocity of blood within a vessel of a patient, pressure data, voltage measurements from an electrode, resistance and/or pressure measurements from a strain sensor, or any other type of data.

The user input device 204 may be any suitable device. For example, the user input device 204 may be configured to receive a user input via one or more buttons or mouse clicks. The user input device 204 may additionally be configured to receive a user input via any other method. For example, the user input device 204 may receive a user input via a touch on a touch screen, an auditory input such as speech or other sounds. In some embodiments, the user input device 204 may be a keyboard, a mouse, a touch screen, one or more buttons, a microphone, or any other suitable device configured to receive inputs from a user.

The processor circuit 206 may be configured to generate, receive, and or process any various data. For example, the processor circuit 206 may be in communication with the memory storage system of the control system 230. The processor circuit 206 may be configured to execute computer readable instructions stored on the memory storage system of the control system 230. The processor circuit 206 may additionally be configured to generate outputs based on any suitable computer readable instructions the circuit 206 may execute. For example, the processor circuit 206 may generate an output configured to be received by a data acquisition device, such as the data acquisition device 224, to begin to receive data. Similarly, the processor circuit 206 may generate an output to be received by a blood flow alteration device, instructing the blood flow alteration device to begin to alter blood flow. In some embodiments, the processor circuit 206 may be further configured to process data received from the devices with which the control system 230 is in communication. In some embodiments, the processor circuit 206 may be configured to generate one or more graphical user interfaces to be output to a display, such as the display 208. In some embodiments, the processor circuit 206 may be additionally configured to receive user inputs from a user input device, such as the user input device 204.

The display 208 may be any suitable display. The display 208 may also be any suitable device. For example, the display 208 may include one or more pixels configured to display regions of an image to a user of the system 200. The display 208 may be in communication with the processor circuit 206 of the control system 230. In this way, the display 208 may receive instructions and/or images to display to a user of the system 200. In some embodiments, the display 208 may show a user a view of the data received and/or processed by the processor circuit 206. The display 208 may additionally convey various recommended actions or prompts for the user of the system 200 from the processor circuit 206. In some embodiments, the display 208 may additionally or alternatively be a user input device. For example, the user of the system 200 may select various elements within a graphic shown on the display 208 to direct the processor circuit 206 of the control system 230 to perform various actions or commands.

The data acquisition subsystem 201 may be in communication with the processor circuit 206, as shown in FIG. 2. The data acquisition subsystem 201 may be any suitable device, system, or subsystem. For example, the data acquisition subsystem 201 may be configured to receive commands from the processor circuit 206 of the control system 230 and send these commands or signals to one or more devices, such as the endovascular device 202. In some embodiments, the data acquisition subsystem 201 may process signals received from the processor circuit 206. In this way, the data acquisition subsystem 201 may facilitate communication between the processor circuit 206 and a device, such as the endovascular device 202. In some embodiments, the data acquisition subsystem 201 may be configured to control a data acquisition device 224. In this way, the data acquisition subsystem 201 and the data acquisition device 224 may together form a data acquisition device system. The data acquisition subsystem 201 may be configured to pre-process the data received from the data acquisition device 224. For example, the data acquisition subsystem 201 may smooth, average, or perform any other suitable preprocessing functions on the data received. The data acquisition subsystem 201 may then be configured to transmit the data received by the data acquisition device 224, which optionally may be preprocessed by the subsystem 201, to the processor circuit 206.

The blood flow alteration subsystem 251 may be configured to control one or more blood flow alteration devices. For example, the blood flow alteration device may be the endovascular device 202. In some embodiments, the endovascular device 202 may include elements of a device configured to alter the blood flow within a renal artery of a patient. For example, the endovascular device 202 may include one or more balloons configured to be positioned within the renal artery and configured to restrict blood flow within the artery when inflated or partially inflated. In another example, the endovascular device 202 may include a pump configured to be positioned within the renal artery and configured to alter blood flow within the renal artery. In some embodiments, and as shown in FIG. 2 the endovascular device 202 may include both a data acquisition device 224 and a blood flow alteration device 254. In this way, the endovascular device 202 may be configured to both receive data and alter blood flow. The blood flow alteration subsystem 251 may be configured to receive command signals from the processor circuit 206. For example, in response to a user input from the user of the system 200, or in response to other computer readable instructions, the processor circuit 206 may generate a command for the blood flow alteration subsystem 251 to begin to alter blood flow, for example, a balloon or a pump. In such an embodiment, the blood flow alteration subsystem 251 may receive such a command from the processor circuit 206 and may generate one or more electrical pulses or electrical signals and transmit these pulses or signals to the device 254. Similarly, the processor circuit 206 may transmit a command to the blood flow alteration subsystem 251 to stop stimulating the nerves of a blood vessel. For example, the processor circuit 206 may generate a command to stop altering blood flow.

As shown in FIG. 2, the endovascular device 202 may be a single device configured to perform multiple functions. However, as will be described in greater detail hereafter, in some embodiments, a data acquisition device, such as the data acquisition device 224 may be housed on a separate device from the device containing the blood flow alteration device.

As shown in FIG. 2, the data acquisition subsystem 201 and the blood flow alteration subsystem 251 may be separate subsystems. In some embodiments, the data acquisition subsystem 201 may be in communication with the data acquisition device 224 of the endovascular device 202. Similarly, the blood flow alteration subsystem 251 may be in communication with the blood flow alteration device 254 of the same endovascular device 202. However, in some embodiments, the data acquisition subsystem 201 and the blood flow alteration subsystem 251 may be the same subsystem. For example, this combined subsystem may be configured to both send and receive data or commands related to the acquisition of data and additionally send and receive commands related to the blood flow alteration device 254.

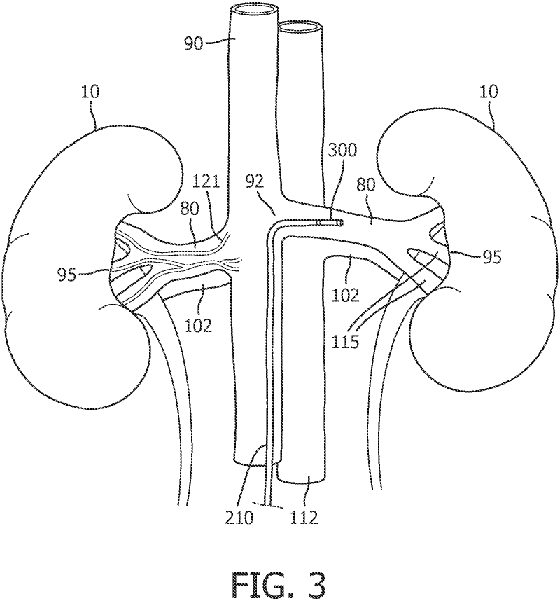

FIG. 3 illustrates an intravascular device 210 disposed within the human renal anatomy. The human renal anatomy includes kidneys 10 that are supplied with oxygenated blood by right and left renal arteries 80, which branch off an abdominal aorta 90 at the renal ostia 92 to enter the hilum 95 of the kidney 10. The abdominal aorta 90 connects the renal arteries 80 to the heart (not shown). Deoxygenated blood flows from the kidneys 10 to the heart via renal veins 102 and an inferior vena cava 112. Specifically, a flexible elongate member of the intravascular device 210 is shown extending through the abdominal aorta and into the left renal artery 80. In alternate embodiments, the intravascular device 210 may be sized and configured to travel through the inferior renal vessels 115 as well. Specifically, the intravascular device 210 is shown extending through the abdominal aorta and into the left renal artery 80. In alternate embodiments, the catheter may be sized and configured to travel through the inferior renal vessels 115 as well.

Left and right renal plexi or nerves 121 surround the left and right renal arteries 80, respectively. Anatomically, the renal nerve 121 forms one or more plexi within the adventitial tissue surrounding the renal artery 80. For the purpose of this disclosure, the renal nerve is defined as any individual nerve or plexus of nerves and ganglia that conducts a nerve signal to and/or from the kidney 10 and is anatomically located on the surface of the renal artery 80, parts of the abdominal aorta 90 where the renal artery 80 branches off the aorta 90, and/or on inferior branches of the renal artery 80. Nerve fibers contributing to the plexi arise from the celiac ganglion, the lowest splanchnic nerve, the corticorenal ganglion, and the aortic plexus. The renal nerves 121 extend in intimate association with the respective renal arteries into the substance of the respective kidneys 10. The nerves are distributed with branches of the renal artery to vessels of the kidney 10, the glomeruli, and the tubules. Each renal nerve 121 generally enters each respective kidney 10 in the area of the hilum 95 of the kidney, but may enter the kidney 10 in any location, including the location where the renal artery 80, or a branch of the renal artery 80, enters the kidney 10.

Proper renal function is essential to maintenance of cardiovascular homeostasis so as to avoid hypertensive conditions. Excretion of sodium is key to maintaining appropriate extracellular fluid volume and blood volume, and ultimately controlling the effects of these volumes on arterial pressure. Under steady-state conditions, arterial pressure rises to that pressure level which results in a balance between urinary output and water and sodium intake. If abnormal kidney function causes excessive renal sodium and water retention, as occurs with sympathetic overstimulation of the kidneys through the renal nerves 121, arterial pressure will increase to a level to maintain sodium output equal to intake. In hypertensive patients, the balance between sodium intake and output is achieved at the expense of an elevated arterial pressure in part as a result of the sympathetic stimulation of the kidneys through the renal nerves 121. Renal denervation may help alleviate the symptoms and sequelae of hypertension by blocking or suppressing the efferent and afferent sympathetic activity of the kidneys 10.

In some embodiments, the vessel 80 is a renal vessel and the pulse wave velocity is determined in the renal artery. The processing system 230 may determine various physiological parameters, such as the blood pressure, blood flow, blood flow velocity, pulse wave velocity (PWV), strain or constriction of the vessel, voltage measurements of renal nerves, or any other parameters in the renal artery. The processing system 230 may determine a renal denervation therapy recommendation based on these parameters in a renal artery. For example, patients that are more likely or less likely to benefit therapeutically from renal denervation may be selected based on the parameters measured. In that regard, based on these parameters measured corresponding to the renal vessel, the processing system 230 can perform patient stratification for renal denervation.

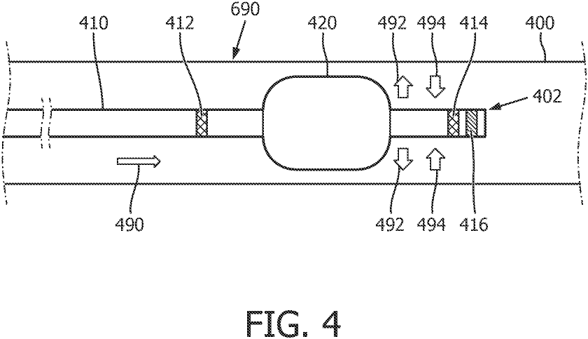

FIG. 4 is a schematic diagram of an endovascular device 402, according to aspects of the present disclosure. The device 402 may be one embodiment of the device 202 described with reference to FIG. 2. As shown in FIG. 4, the device 402 may be configured to be positioned within a blood vessel 400 of a patient. For example, as shown in FIG. 4, a diagrammatic view of a blood vessel 400 is provided. The device 202 may include a flexible elongate member 410, a balloon 420, a proximal pressure sensor 412, a distal pressure sensor 414, and a blood flow sensor 416. In some aspects, any of the sensors 412, 414, or 416 may be other suitable type of sensors used to obtain a metric related to the blood vessel. For example, any of the sensors 412, 414, or 416 may alternatively be a pressure sensor, a flow sensor, an electrode, a strain sensor, or any other suitable type of sensor.

The flexible elongate member 410 may be sized and shaped, structurally arranged, and/or otherwise configured to be positioned within a body lumen 400 of a patient. The flexible elongate member 410 may be a part of guidewire and/or a catheter (e.g., an inner member and/or an outer member). The flexible elongate member 410 may be constructed of any suitable flexible material. For example, the flexible elongate member 410 may be constructed of a polymer material including polyethylene, polypropylene, polystyrene, or other suitable materials that offer flexibility, resistance to corrosion, and lack of conductivity. In some embodiments, the flexible elongate member 410 may define a lumen for other components to pass through. The flexible elongate member 410 may be sufficiently flexible to successfully maneuver various turns or geometries within the vasculature of a patient. The flexible elongate member 410 may be of any suitable length or shape and may have any suitable characteristics or properties.

The balloon 420 may include a device configured to expand and contrast in a radial direction. For example, in some embodiments, the balloon 420 may be filled with a liquid or gaseous substance. As the balloon 420 is filled, the outermost walls of the balloon 420 may expand radially outward as shown by the arrows 492 in FIG. 4. By contrast, when the liquid or gaseous substance is removed from the balloon 420, the outer walls may contract in a radially inward direction as shown by the arrows 494 of FIG. 4.

In this way, the balloon 420 may alter the blood flow within the renal artery 400. For example, blood may move through the renal artery in a direction shown by the arrow 490. In an expanded position, the balloon 420 may block the flow of blood. For example, when the balloon 420 is fully expanded, it may come into contact with the vessel walls of the renal artery 400 and completely cut off blood flow. With the balloon 420 partially inflated, blood may be allowed to flow around the balloon 420 in the direction of the arrow 490, but the flow may be partially inhibited.

In some embodiments, the flow sensor 416 positioned distal to the balloon 420 may determine the flow of blood. Based on the flow information received by the flow sensor 416, a user of the system 200, or a processor circuit (e.g., the processor circuit 206) may adjust the inflation of the balloon 420. For example, the balloon 420 may be inflated to a point that a target flow value is measured by the sensor 416. The target flow value may be input by the user, based on expert recommendations, or input in any other way.

The pressure sensor 414 may acquire pressure measurements distal of the balloon 420. The pressure measurements received from the pressure sensor 414 may also be used to determine the extent of inflation of the balloon 420.

In some embodiments, the pressure sensor 414 and the flow sensor 416 may both be used to determine the extent of inflation of the balloon 420. In other embodiments, only the pressure sensor 414 or the flow sensor 416 may be used to determine the extent of inflation.

In some embodiments, the flow sensor 416 may be positioned proximal to the balloon 420. In such an embodiment, the physician may ensure that the balloon and the flow sensor 416 do not cross a side branch of the vessel 400.

Further illustrated in FIG. 4 is the proximal pressure sensor 412. As the balloon 420 is inflated and blood flow is restricted within the renal artery 400, the sympathetic drive of the renal nerve (e.g., the nerves 121 of FIG. 3) may be altered. For example, the renal nerves may cause the renal artery 400 to expand or contract in response to reduced blood flow. Other physiological responses may occur. In some embodiments, the restriction in blood flow caused by the expansion of the balloon 420 may cause the blood pressure of the patient to decrease. The proximal sensor 412 may monitor the blood pressure of the patient to identify and quantify this response to the decreased blood flow. In some embodiments, the flow sensor 416 may be a thermoelectric sensor.

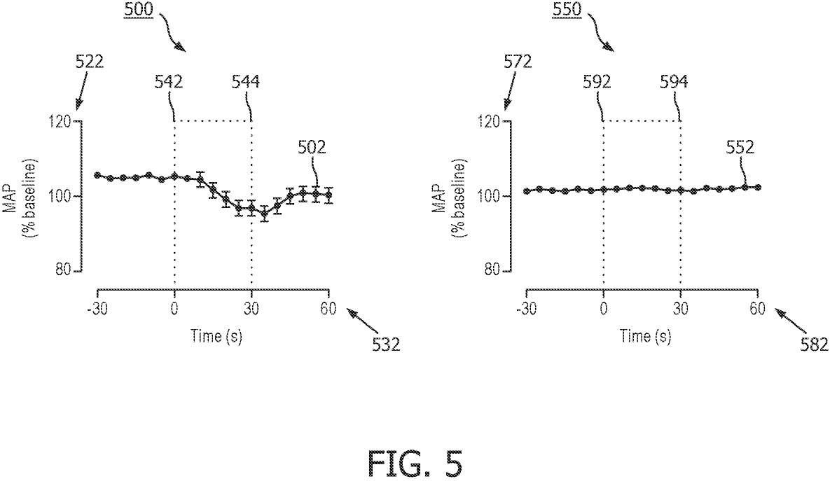

FIG. 5 is a diagrammatic view of hemodynamic data associated with a blood flow alteration procedure, according to aspects of the present disclosure. The hemodynamic data may include a plot 500 and a plot 550.

In one embodiment, the plot 500 may correspond to hemodynamic measurements of a renal artery before a renal denervation procedure is performed and the plot 550 may correspond to hemodynamic measurements of the same renal artery after a renal denervation procedure is performed. In such an embodiment, the plot 500 and the plot 550 may be acquired by the same endovascular device (e.g., the device 202 shown in FIG. 4) or any other devices described hereafter. As shown in FIG. 5, a user of the system 200 and/or a processor circuit may determine that a renal denervation procedure was successful based on a comparison of the data. As shown in the plot 500, a decrease in blood pressure was observed during the time period of blood flow alteration. This blood pressure may be measured by the proximal pressure sensor 412. However, in the plot 550, no significant decrease in blood pressure was observed. This little to no change in blood pressure in response to a decrease in blood flow in the renal artery (e.g., caused by the expanded balloon 420 or another device as described hereafter) may indicate that the renal denervation procedure was successful, and that hypertension may be relieved with the ablation of the renal nerves.

In one embodiment, the plot 500 may correspond to hemodynamic measurements altering the blood flow at one location along the renal artery or within one single side branch (e.g., such as one of the vessels 115 of FIG. 3) and the plot 550 may correspond to hemodynamic measurements of a different location or side branch. The plot 500 and plot 550 may correspond to data acquired by the same device at a different location along the same renal artery. In this way, the user of the system 200 may target specific locations or side branches (e.g., vessels 115 of FIG. 3) to ablate during a renal denervation procedure based on the response to alteration of blood flow at these respective locations. For example, the plot 500 may correspond to a side branch 115 that is likely to respond well to renal denervation in decreasing hypertension, while the plot 550 may correspond to a side branch 115 which will not respond well to renal denervation in decreasing hypertension.

In another embodiment, the plot 500 may correspond to hemodynamic measurements of one patient and the plot 550 may correspond to hemodynamic measurements of a different patient. For example, the plot 500 may correspond to a patient that is likely to respond well to renal denervation in decreasing hypertension, while the plot 550 may correspond to a patient which will not respond well to renal denervation in decreasing hypertension. In this way, the system 200 may help a physician stratify patients who are likely to be aided by a renal denervation procedure and patients who are likely not to be aided by a renal denervation procedure.

The plots 500 and 550 may include any suitable hemodynamic data, for example blood pressure. However, other data may include blood flow, resistance of blood flow, electrical resistance along a vessel, voltage associated with renal nerves, resistance or pressure of a strain sensor, or any other data. In the embodiment shown, the plots 500 and 550 may correspond to a mean arterial pressure (MAP) of the blood within the patient vasculature. In some aspects, the plots 500 and 550 may be displayed to a user via the display (e.g., the display 208). In some aspects, the display shown to a user may include any of the data points of the plots 500 and 550. For example, the display may include a data point obtained when the patient’s sympathetic nervous system is not under stimulation (e.g., a data point obtained prior to the time shown by the line 542) and a data point obtained when the patient’s sympathetic nervous system is under stimulation (e.g., a data point obtained between the line 542 and 544).

The plot 500 may include an axis 522. The axis 522 may define a scale associated with blood pressure measurements. The MAP axis 522 may provide a visual illustration of mean blood pressure measurements within the renal artery. For example, it may provide a reference such that locations of blood pressure measurements may indicate the corresponding value. The range of the MAP axis 522 may be automatically adjusted by the processor circuit of the system 100 or may be adjusted by a user. The plot 550 includes a similar axis 572.

The plot 500 may additionally correspond to a time axis 532. The time axis 532 shown in FIG. 5 may illustrate elapsed time of a procedure. Any region of the time axis 532 may correspond to any time of the procedure. The time axis 532 may be continuously shifted so as to display the time of the most recent measurement and an arbitrary number of previous times as well. For example, the time axis 532 may be updated according to a live measurement procedure such that a user of the system may observe measurements within the plot 500 as they are acquired. The plot 550 includes a similar axis 582.

The plot 500 may additionally include multiple MAP data points 502. Each MAP data point 502, or blood pressure data point, may include a two-coordinate data point including a MAP measurement value and a time value. The MAP data points may be referred to as blood pressure metrics. In some aspects, a blood pressure metric may be a blood pressure value, measured in mmHg or any other suitable unit, or may be a fractional flow reserve (FFR) value, instantaneous wave-free ratio (iFR) value, a pressure ratio, or any other suitable values. The MAP measurement value may correspond to the blood pressure measured by a pressure sensor (e.g., the proximal pressure sensor 412). The time value may correspond to the time along the time axis 532 at which the associated blood pressure measurement was acquired. In this way, the data points 502 may be positioned within the plot 500 so as to correspond to the pressure value and the time value. Similarly, the plot 550 may include multiple MAP data points 552.

The plot 500 includes a dotted line 542. The line 542 may be a vertical line corresponding to a time measurement. In one embodiment, the line 542 may correspond to the time at which a blood flow alteration device (e.g., the balloon 420) began to alter blood flow (e.g., expand). The line 542 may be of any suitable visual appearance. The data 502 of the plot 500 may be data acquired by the intravascular device while the intravascular device is positioned within the blood vessel.

An additional dotted line 544 is also shown. The line 544 may be a vertical line corresponding to a time measurement and may be overlaid over all the plot 500. The line 544 may correspond to the time at which the blood flow alteration device stopped altering the blood flow. The line 544 may be similar to the line 542 in that it may be of any suitable appearance. The plot 550 includes similar lines 592 and 594 denoting the start and stop of blood flow alterations.

It is additionally noted that all percentage or other values described herein are merely exemplary and for pedagogical purposes only. Any suitable values including percentages of baseline values of hemodynamic parameters may be contemplated.

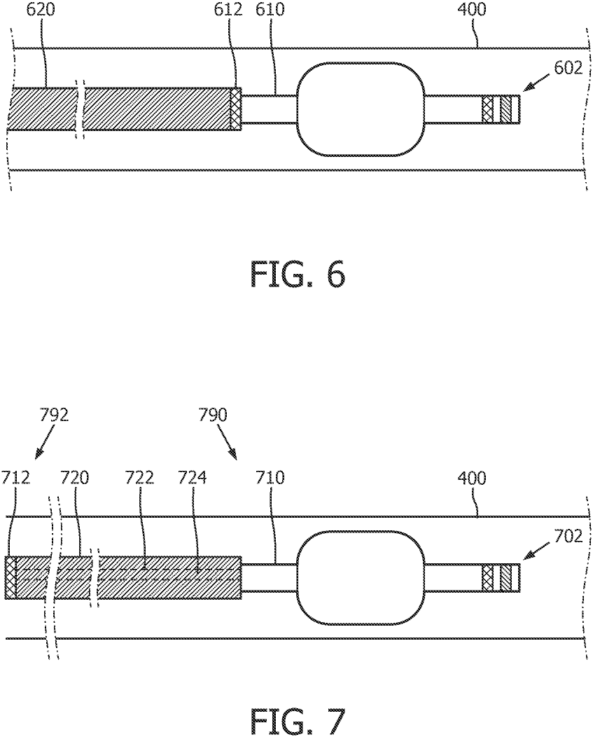

FIG. 6 is a schematic diagram of an endovascular device 602, according to aspects of the present disclosure. The device 602 may be one embodiment of the device 202 described with reference to FIG. 2. As shown in FIG. 6, the device 602 may be configured to be positioned within the blood vessel 400 of a patient. The vessel 400 may be a renal artery of a patient.

Various aspects of the device 602 may be similar to the device 402. However, the device 602 may include a guide catheter 620. In some embodiments, the guide catheter 620 may alternatively be referred to as an introducer. The guide catheter 620 may include a central lumen sized and shaped to receive a flexible elongate member 610. The balloon and distal pressure sensor and flow sensor may be positioned on the flexible elongate member 610.

At a distal portion of the guide catheter 620, a pressure sensor 612 may be positioned. In some embodiments, the pressure sensor 612 shown in FIG. 6 may perform the same function as the pressure sensor 412 of FIG. 4. For example, the pressure sensor 612 may acquire pressure measurements during a blood flow alteration procedure. In some embodiments, the pressure measurements from the pressure sensor 612 may be used to assess the likelihood of success of a planned renal denervation procedure or the degree of success of a completed renal denervation procedure, as described with reference to FIG. 5. In some embodiments, the proximal sensor 612 may be positioned within the renal artery 400. In some embodiments, the pressure sensor 612 may be positioned within the aorta of a patient while the balloon and distal sensors are positioned in the renal artery. In other embodiments, the sensor 612 may be positioned within any suitable vessel of the patient.

FIG. 7 is a schematic diagram of an endovascular device 702, according to aspects of the present disclosure. The device 702 may be one embodiment of the device 202 described with reference to FIG. 2. As shown in FIG. 7, the device 702 may be configured to be positioned within the blood vessel 400 of a patient.

Various aspects of the device 702 may be similar to the device 402. However, the device 702 may include a guide catheter 720. In some embodiments, the guide catheter 720 may alternatively be referred to as an introducer or a sheath. The guide catheter 720 may include a central lumen sized and shaped to receive a flexible elongate member 710. The balloon and distal pressure sensor and flow sensor may be positioned on the flexible elongate member 710. The guide catheter 720 may include a large bore or long tube to protect the artery when you put other devices through. It may also include an infusion line which may be used to inject fluids into the vessel.

The guide catheter may also include an additional lumen 722. The lumen 722 may extend along a central portion of the guide catheter 720 from a position 792 outside the patient’s body to a distal position 790. The lumen 722 may be filled with a fluid 724. The fluid 724 may be blood from the vessel 400 which may enter at an opening in the guide catheter 720 at the position 790. In some embodiments, the fluid 724 may be a saline solution, or other fluid including various medications which may be introduced to the vessel 400 via the lumen 722. In some embodiments, a barrier may separate the fluid 724 from the blood of the vessel.

At a proximal portion of the guide catheter 720, at the position 792 outside the body, a proximal pressure sensor 712 may be positioned. In some embodiments, the pressure sensor 712 shown in FIG. 7 may perform the same function as the pressure sensor 412 of FIG. 4 or the sensor 612 of FIG. 6. For example, the pressure sensor 712 may acquire pressure measurements during a blood flow alteration procedure. In some embodiments, the pressure of the fluid 724 within the lumen 722 may be measured by the pressure sensor 712. Because the pressure of the fluid 724 may be the same as the blood within the lumen 400, the pressure of the blood within the vessel 400 may be measured by the pressure sensor 712. In this way, although the pressures sensor 712 is located at the location 792, the sensor 712 may measure pressure within the renal artery 400 at the location 790. In some embodiments, the lumen 722 may be filled with a fluid that is not the blood of the patient. For example, a barrier may separate blood of the patient from the fluid within the lumen 722 at the location 790.

In some embodiments, the pressure measurements from the pressure sensor 712 may be used to assess the likelihood of success of a planned renal denervation procedure or the degree of success of a completed renal denervation procedure, as described with reference to FIG. 5. In some embodiments, the distal portion of the guide catheter may be positioned within the renal artery 400. In some embodiments, the distal portion of the guide catheter may be positioned within the aorta of a patient while the balloon and distal sensors are positioned in the renal artery. In other embodiments, the distal portion of the guide catheter may be positioned within any suitable vessel of the patient.

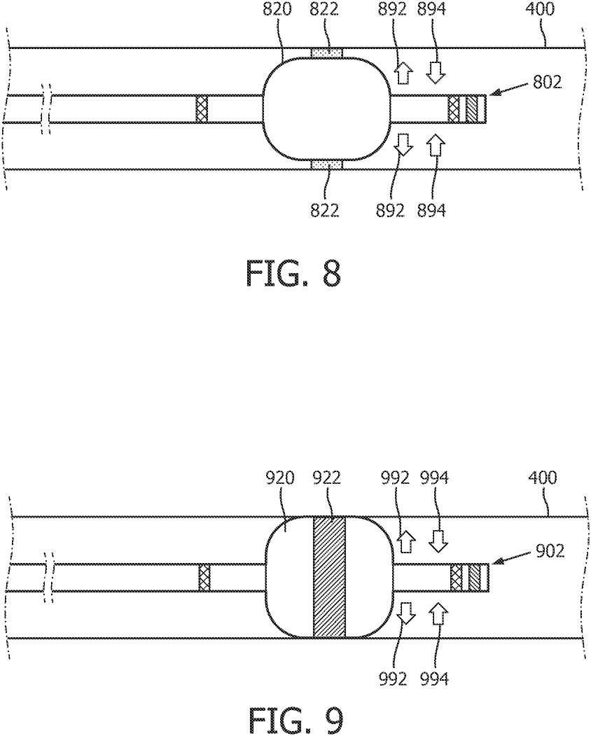

FIG. 8 is a schematic diagram of an endovascular device 802, according to aspects of the present disclosure. The device 802 may be one embodiment of the device 202 described with reference to FIG. 2. As shown in FIG. 8, the device 802 may be configured to be positioned within the blood vessel 400 of a patient.

The device 802 shown in FIG. 8, like the devices previously described may include structures configured to alter blood flow within the renal artery 400 as well as structures configured to monitor the sympathetic response to the reduction in blood flow.

As shown, the device 802 may include a balloon 820. The balloon 820 may be similar to the balloon 420 described with reference to FIG. 4. However, in the embodiment shown in FIG. 8, multiple electrodes 822 may be positioned on an outer surface of the balloon 820. The electrodes 822 may be a part of a data acquisition device (e.g., the device 224 of FIG. 2). For example, as the balloon 820 is expanded and blood flow through the renal artery 400 is restricted, the renal nerves (e.g., the nerves 121 of FIG. 3) may send and/or receive neural impulses from and/or to the central nervous system. As a result, the artery 400, as well as other arteries within the patient, may expand or contrast. In one embodiment, the renal artery 400, in response to a neural signal associated with the renal artery 400 may contract, lowering the blood pressure in the patient. As previously described, the response of the sympathetic nervous system to the restriction of blood flow by the balloon may be identified by a drop in pressure as observed by a proximal pressure sensor (e.g., the sensor 412, 612, and/or 712). However, the electrodes 822 may also identify a response of the sympathetic nervous system by detecting changes in potential or voltage within the renal nerves, corresponding to a neural impulse being sent or received. This data may be used to identify whether a patient is likely to respond positively to a renal denervation procedure, whether a particular side branch or other location is a good location for renal denervation, or whether a renal denervation procedure was successful.

As an example, referring again to FIG. 5, plots similar to the plots 500 and 550 may be received including data from the electrodes 822. However, rather than mean arterial pressure being shown by the plots, a voltage may be displayed. If a change in voltage is observed in response to a decrease in blood flow (e.g., like the change in pressure observed in the plot 500), the user of the system 200 or a processor circuit may determine that the patient/location is a good candidate for renal denervation. Alternatively, if the plot is obtained after a renal denervation procedure, the renal denervation procedure may have been unsuccessful. However, if little to no change in voltage is detected by the electrodes 822, a plot similar to the plot 550 may result. In this case, the patient/location may not be ideal for a renal denervation procedure. Alternatively, if the plot is obtained after a renal denervation procedure, the renal denervation procedure may have been successful.

It is noted that in other embodiments, the structure 820 may not be a balloon. For example, in some embodiments, the device 802 may be used only to monitor sympathetic response and may not be configured to alter blood flow. In such an embodiment, no balloon may be included. Rather, a separate device which may move the electrodes in radial directions (e.g., as shown by the arrows 892 and 894) may replace the balloon. For example, the structure 820 may alternatively be a basket catheter. In such an embodiment, the structure 820 may not inhibit or alter blood flow but may allow blood to pass through the structure freely. Aspects of the structure 820 may include features described in U.S. Pat. Application 13/458,856 (Atty. Docket No. 2012P02290US / 44755.805US01), titled, “METHODS AND APPARATUS FOR RENAL NEUROMODULATION” and filed Apr. 27, 2012, which is hereby incorporated by reference in its entirety.

FIG. 9 is a schematic diagram of an endovascular device 902, according to aspects of the present disclosure. The device 902 may be one embodiment of the device 202 described with reference to FIG. 2. As shown in FIG. 9, the device 902 may be configured to be positioned within the blood vessel 400 of a patient.

The device 902 shown in FIG. 9, like the devices previously described may include structures configured to alter blood flow within the renal artery 400 as well as structures configured to monitor the sympathetic response to the reduction in blood flow.

As shown, the device 902 may include a balloon 920. The balloon 920 may be similar to the balloon 420 described with reference to FIG. 4. However, in the embodiment shown in FIG. 9, a strain sensor 922 may be positioned within, or on a surface of, the balloon 920. The strain sensor 922 may be a part of a data acquisition device (e.g., the device 224 of FIG. 2). For example, as the balloon 920 is expanded and blood flow through the renal artery 400 is restricted, the renal nerves (e.g., the nerves 121 of FIG. 3) may send and/or receive neural impulses from and/or to the central nervous system. As a result, the artery 400, as well as other arteries within the patient, may expand or contrast. In one embodiment, the renal artery 400, in response to a neural signal associated with the renal artery 400 may contract, lowering the blood pressure in the patient. As previously described, the response of the sympathetic nervous system to the restriction of blood flow by the balloon may be identified by a drop in pressure as observed by a proximal pressure sensor (e.g., the sensor 412, 612, and/or 712). However, the strain sensor 922 may also identify a response of the sympathetic nervous system by detecting changes in the shape or tone of the walls of the renal artery 400. For example, regions of the strain sensor may be brought into contact with the walls of the vessel 400, as shown. While in contact with the wall, the strain sensor may measure to what extent the vessel walls contract or expand, as well as with what pressure the vessel wall pushes against the strain sensor. The greater the strain measured by the strain sensor 922, the greater the sympathetic response to the blood flow restriction. This data may be used to identify whether a patient is likely to respond positively to a renal denervation procedure, whether a particular side branch or other location is a good location for renal denervation, or whether a renal denervation procedure was successful.

As an example, referring again to FIG. 5, plots similar to the plots 500 and 550 may be received including data from the strain sensor 922. However, rather than mean arterial pressure being shown by the plots, a resistance or pressure may be displayed. If a change in resistance or pressure is observed in response to a decrease in blood flow (e.g., like the change in pressure observed in the plot 500), the user of the system 200 or a processor circuit may determine that the patient/location is a good candidate for renal denervation. Alternatively, if the plot is obtained after a renal denervation procedure, the renal denervation procedure may have been unsuccessful. However, if little to no change in resistance or pressure is detected by the strain sensor 922, a plot similar to the plot 550 may result. In this case, the patient/location may not be ideal for a renal denervation procedure. Alternatively, if the plot is obtained after a renal denervation procedure, the renal denervation procedure may have been successful.

Similar to the structure 820 of FIG. 8, the structure 920 may not be a balloon, but may be another structure which allows blood flow even while the strain sensor is brought in contact with the vessel walls.

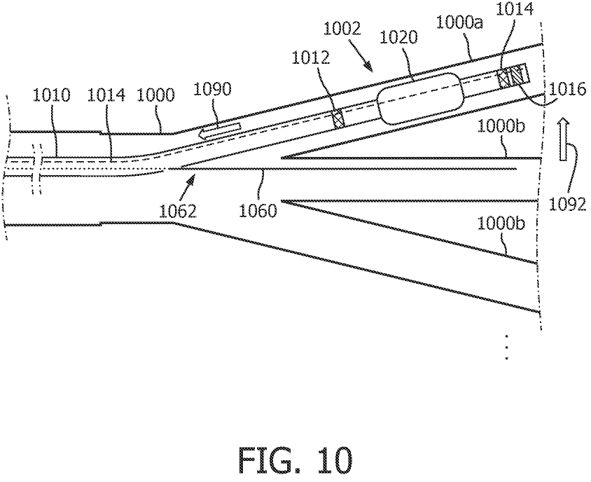

FIG. 10 is a schematic diagram of an endovascular device 1002, according to aspects of the present disclosure. The device 1002 may be one embodiment of the device 202 described with reference to FIG. 2. As shown in FIG. 10, the device 902 may be configured to be positioned within a blood vessel of a patient.

The device 1002 shown in FIG. 10, like the devices previously described may include structures configured to alter blood flow within the renal artery as well as structures configured to monitor the sympathetic response to the reduction in blood flow.

A renal artery 1000 is shown in FIG. 10. The renal artery 1000 may, at a distal end, split into multiple side branches. For example, a side branch 1000a, a side branch 1000b, and a side branch 1000c are shown. It is noted that additional or fewer side branches may be included within the renal vasculature.

In the embodiment shown, a portion of the device 1002 may be positioned within one side branch (e.g., the side branch 1000a) while a separate portion of the device 1002 may be positioned within a different side branch (e.g., the side branch 1000b). In some embodiments, the measurement portion of the device 1002 (e.g., a proximal pressure sensor 1012, a distal pressure sensor 1014, a distal flow sensor 1016, and/or a balloon 1020) may be moved to different side branches within the renal vasculature without completely removing the device 1002.

As shown in FIG. 10, a guidewire 1060 may extend along the longitudinal center of the device 1002. In some embodiments, the guidewire 1060 may be positioned within the renal artery first. In the embodiment shown, the guidewire 1060 may be positioned within the side branch 1000b. The device 1002 may then be positioned around the guidewire 1060. For example, a lumen of the device 1002 may be sized to receive the guidewire 1060. At the opening 1062, the device 1002 may be positioned around the guidewire 1060. The device 1006 may then move along the guidewire through the patient vasculature to the renal vasculature. There, the device 1002 may be positioned within the same side branch 1000b with the guidewire 1060. After measurements are made there, however, the device 1002 may be moved in a proximal direction so as to exit the side branch 100b and return to the primary renal artery 1000. There, the measurement portion of the device 1002 may be deflected from the guidewire 1060 so as to be positioned in a separate side branch (e.g., the side branch 1000a) while the guidewire 1060 remains in the same side branch (e.g., the side branch 1000b).

In some embodiments, the device may include one or more pull wires 1014. A pull wire (e.g., the pull wire 1014) may be positioned within the device 1002 or on an outer surface of the device 1002. In some embodiments, the pull wire 1014 may be attached to a side of the device 1002 or a side of the flexible elongate member 1010 of the device 1002. In this way, when a physician, or other automated or robotic system, pulls on the pull wire 1014, a force is exerted in the proximal direction shown by the arrow 1090. Due to the flexible nature of the device 1002, this force on one side of the device 1002 causes the device to deflect away from the guidewire 1060 in a direction corresponding the to the location at which the pull wire 1014 is attached to the device.

FIG. 11 is a schematic diagram of an endovascular device 1102, according to aspects of the present disclosure. The device 1102 may include elements corresponding to blood flow alteration as well as elements corresponding to data acquisition. The device 1102 may be an embodiment of the device 202 (FIG. 2).

In the embodiment shown in FIG. 11, the blood flow alteration device (e.g., the blood flow alteration device 254 of FIG. 2), may include an on-board pump 1130 and a corresponding lumen 1160. Like the balloon described with reference to previous figures, the pump 1130 may alter the blood flow within the renal artery 1100. As shown in FIG. 11 and FIG. 12, the pump may be coupled to the intravascular device (e.g., a proximal end, as in FIG. 12) or the pump may be disposed or integrated within the intravascular device (as in FIG. 11).

In some embodiments, the on-board pump 1130 may be positioned within the device 1102, as shown. An opening 1162 may be positioned within the device 1102 distal of the pump 1130 and an additional opening 1164 may be positioned within the device 1102 proximal to the pump 1130. As shown, blood from within the renal artery 1100 may enter the lumen 1160 by the opening 1162. The pump may move blood in a proximal direction from the opening 1162 to the opening 1164. In this way, the pump 1130 may suck blood through the opening 1162, as shown by the arrow 1193, and through the lumen 1160 as shown by the arrow 1194. The pump 1130 may also send blood further along the lumen 1160 as shown by the arrow 1195 and out the opening 1164 as shown by the arrow 1196. In this way, blood that was originally within the renal artery 1100 may be moved back to the aorta 1101. The flow of blood is shown by the arrows 1191 and 1192. In this way, the pump may push blood upstream of the natural flow of blood. After exiting the opening 1164, some blood may flow further downstream of the renal artery as shown by the arrow 1193. In this way, the amount of blood passing to the renal artery is decreased, thus restricting the blood flow within the renal artery 1100 and to the kidney.

The device 1102 may include any suitable sensors. As an example, the device 1102 may include a distal sensor 1114 and a proximal sensor 1112. The distal sensor 1114 may monitor the pressure distal of the pump. In this way, the distal sensor 1114 may assess the effectiveness of the pump 1130 as well as assess the extent to which blood flow has been reduced. In some embodiments, an additional distal sensor may be included. This additional sensor may be a flow sensor. In some embodiments, the distal sensor 1114 may be a flow sensor and no distal pressure sensor may be present distal of the pump 1130.

The proximal pressure sensor 1112 may serve the same purpose as the proximal pressure sensor 412 of FIG. 4 or any of the proximal pressure sensors described previously. In particular, the proximal pressure sensor 1112 may monitor the blood pressure of the patient to assess the sympathetic response to the alteration of blood flow caused by the pump 1130. The data collected from the proximal sensor 1112 may be used to generate plots similar to those described with reference to FIG. 5 and/or may be used to determine whether a patient will be responsive to a renal denervation procedure, whether a location within the renal vasculature will be responsive to a renal denervation procedure, or whether a renal denervation procedure was successful.

It is noted that the pump 1130 may be engaged to move fluids in the opposite direction than as shown. For example, the pump 1130 may move blood from the opening 1164 in the aorta 1101 to the opening 1162 in the renal artery 1100. In this way, the pump 1130 may be used to increase blood flow. Similar, but opposite changes in blood pressure may then be monitored by the pressure sensor 1112.

It is also noted that the device 1102 may be positioned within the renal artery 1100 in any way. For example, the device 1102 may be inserted into the patient via a femoral artery and may approach the renal artery from below. In this way, blood pumped from the renal artery 1100 may be moved to the aorta at some point below, or downstream of, the renal artery, such as at the location 1197 shown. In such an embodiment, blood exiting the opening 1164 may not return into the renal artery 1100, but all the blood exiting the opening 1164 may proceed downstream and away from the renal artery. In some embodiments, this orientation of the device 1102 may increase the effect of the pump 1130 in decreasing blood flow within the renal artery 1100. In some embodiments, the pressure sensor 1112 may be positioned distal to the opening 1164. The proximal pressure sensor 1112 may acquire more accurate pressure measurements if positioned upstream of the opening 1164 whether the device 1102 is in the configuration shown in FIG. 11 or in the configuration described in the present paragraph with entry from below, such as through a femoral artery.

FIG. 12 is a schematic diagram of an endovascular device 1202, according to aspects of the present disclosure. The device 1202 may include elements corresponding to blood flow alteration as well as elements corresponding to data acquisition. The device 1202 may be an embodiment of the device 202 (FIG. 2). The device 1202 may be similar to the device 1102 except that the pump of the device 1202 may be external.

Specifically, a view of the same renal artery 1100 and aorta 1101 are provided. The device 1202 may include a distal sensor 1214 similar to the sensor 1114 and a proximal sensor 1212 similar to the sensor 1112. The device 1202 may include at least two lumens: a lumen 1260 extending from the external pump 1230 to an opening 1262 and a lumen 1270 extending from the external pump 1230 to an opening 1272. In some embodiments, the device 1202 may move blood from the renal artery 1100 to the aorta 1101, like the device 1102.

When engaged, the external pump 1230 may draw blood from the renal artery 1100 through the opening 1262, as shown by the arrow 1293, and along the lumen 1260 in a proximal direction, as shown by the arrow 1294. As shown by the arrow 1295, the blood may pass into the pump 1230, at which point it is sent out of the pump, as shown by the arrow 1297. The blood may then travel along the lumen 1270 in a distal direction, as shown by the arrow 1298, until it exits via the opening 1272, as shown by the arrow 1296. In this way, the device 1202 may alter the blood flow within the renal artery 1100.

As described with reference to FIG. 11, the sensors 1212 and/or 1214 may monitor blood pressure and/or flow within the vasculature to assess the effectiveness of the pump 1230 as well as the sympathetic effect of the blood flow alteration.

As noted with reference to FIG. 11, the device may be oriented from the bottom such that it enters through a femoral artery. In this case, the position of the sensor 1212 may be positioned upstream of the opening 1272 according to the same principles described with reference to FIG. 11.

It is noted that any of the blood flow alteration devices described herein (e.g., the balloon 420 of FIG. 4, the balloon 820 of FIG. 8, the balloon 920 of FIG. 9, the balloon 1020 of FIG. 10, the pump 1130 of FIG. 11, and/or the pump 1230 of FIG. 12) may be used with any of the data acquisition devices described herein (e.g., the sensors 412, 414, and 416 of FIG. 4, the sensors 612 and 712, the electrodes of the electrodes 822 of FIG. 8, the strain sensor 922 of FIG. 9, the sensors of FIGS. 11 and 12, and/or any additional described data acquisition devices, such as a blood flow resistance device including multiple pressure and flow sensors, intravascular imaging devices, and/or any other devices).

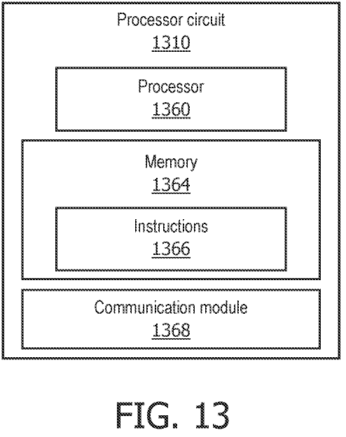

FIG. 13 is a schematic diagram of a processor circuit, according to aspects of the present disclosure. The processor circuit 1310 may be implemented in the control system 230 (e.g., as shown in FIG. 2), or any other suitable location. In an example, the processor circuit 1310 may be in communication with any of the devices, systems, or subsystems described in the present disclosure. For example, the processor circuit 1310 may be in communication with a blood flow sensing device, a pressure sensing device, an extraluminal imaging device, a nerve stimulation device, a nerve ablation device or any other device, system, or subsystem. The processor circuit 1310 may include a processor 106 and/or a communication interface. One or more processor circuits 1310 are configured to execute the operations described herein. As shown, the processor circuit 1310 may include a processor 1360, a memory 1364, and a communication module 1368. These elements may be in direct or indirect communication with each other, for example via one or more buses.

The processor 1360 may include a CPU, a GPU, a DSP, an application-specific integrated circuit (ASIC), a controller, an FPGA, another hardware device, a firmware device, or any combination thereof configured to perform the operations described herein. The processor 1360 may also be implemented as a combination of computing devices, e.g., a combination of a DSP and a microprocessor, a plurality of microprocessors, one or more microprocessors in conjunction with a DSP core, or any other such configuration.

The memory 1364 may include a cache memory (e.g., a cache memory of the processor 1360), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1364 includes a non-transitory computer-readable medium. The memory 1364 may store instructions 1366. The instructions 1366 may include instructions that, when executed by the processor 1360, cause the processor 1360 to perform the operations described herein with reference to any of the devices, system, or subsystems described. Instructions 1366 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1368 can include any electronic circuitry and/or logic circuitry to facilitate direct or indirect communication of data between the processor circuit 1310, the devices, systems, or subsystems described herein, the display 208, processor circuit 206, or user input device 204 (FIG. 2). In that regard, the communication module 1368 can be an input/output (I/O) device. In some instances, the communication module 1368 facilitates direct or indirect communication between various elements of the processor circuit 1310 and/or various described endovascular or extraluminal devices, systems, and/or the host 230 (FIG. 2).

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
an intravascular catheter or guidewire sized and shaped for positioning within a first blood vessel of a patient; and
a processor circuit configured for communication with the intravascular catheter or guidewire, wherein the processor circuit is configured to:
determine, using the intravascular catheter or guidewire, a first metric corresponding to a first state of a sympathetic nervous system of the patient;
control the intravascular catheter or guidewire to alter a blood flow within the first blood vessel;
determine, using the intravascular catheter or guidewire, a second metric corresponding to a second state of the sympathetic nervous system of the patient, the second state of the sympathetic nervous system resulting from the altered blood flow within the first blood vessel; and
provide, to a display in communication with the processor circuit, an output based on the first metric and the second metric.

2. The system of claim 1,
wherein the intravascular catheter or guidewire comprises a blood flow sensor, and
wherein the processor circuit is configured to receive, from the blood flow sensor, blood flow data representative of the blood flow within the first blood vessel.

3. The system of claim 2, wherein the processor circuit is configured to control the intravascular catheter or guidewire to alter the blood flow based on the blood flow data.

4. The system of claim 1,
wherein the intravascular catheter or guidewire comprises a balloon, and
wherein, to control the intravascular catheter or guidewire to alter the blood flow, the processor circuit is configured to control expansion of the balloon within the first blood vessel to restrict the blood flow.

5. The system of claim 1,
wherein the intravascular catheter or guidewire comprises a pump, and
wherein to control the intravascular catheter or guidewire to alter the blood flow, the processor circuit is configured to control the pump to:
move blood from the first blood vessel to a second blood vessel; or
move blood from the second blood vessel to the first blood vessel.

6. The system of claim 1,
wherein the intravascular catheter or guidewire comprises a pressure sensor, and
wherein the first metric comprises a first blood pressure metric and the second metric comprises a second blood pressure metric.

7. The system of claim 1,
wherein the intravascular catheter or guidewire comprises at least one pressure sensor and at least one flow sensor, and
wherein the first metric comprises a first fluid resistance metric and the second metric comprises a second fluid resistance metric.

8. The system of claim 1,
wherein the intravascular catheter or guidewire comprises an electrode, and
wherein the first metric corresponds to a first voltage metric and the second metric corresponds to a second voltage metric.

9. The system of claim 1,
wherein the intravascular catheter or guidewire comprises a strain sensor, and
wherein the first metric corresponds to a first resistance metric and the second metric corresponds to a second resistance metric.

10. The system of claim 1, wherein the processor circuit is configured to perform a comparison based on the first metric and the second metric.

11. The system of claim 10, wherein the comparison comprises a determination of whether a difference between the first metric and the second metric exceeds a threshold difference.

12. The system of claim 10,
wherein the blood vessel is a renal artery,
wherein the comparison comprises a determination of whether a renal denervation is recommended for the patient, and
wherein the output comprises a visual representation of the determination.

13. The system of claim 10,
wherein the blood vessel is a renal artery,
wherein the comparison comprises a determination of whether a renal denervation was successful, and
wherein the output comprises a visual representation of the determination.

14. A method, comprising:
determining, with a processor circuit in communication with an intravascular catheter or guidewire, a first metric corresponding to a first state of a sympathetic nervous system of the patient using an intravascular catheter or guidewire positioned within a blood vessel;
controlling, with the processor circuit, the intravascular catheter or guidewire to alter a blood flow within the blood vessel using the intravascular catheter or guidewire;
determining, with the processor circuit, a second metric corresponding to a second state of the sympathetic nervous system of the patient using the intravascular catheter or guidewire, the second state of the sympathetic nervous system resulting from the altered blood flow within the blood vessel; and
providing, with the processor circuit, an output based on the first metric and the second metric to a display in communication with the processor circuit.

15. A system, comprising:
an intravascular catheter or guidewire sized and shaped to be positioned within a renal artery of a patient, wherein the intravascular catheter guidewire comprises:
one or more sensors; and
at least one of a balloon or a pump; and
a processor circuit configured for communication with the intravascular catheter or guidewire, wherein the processor circuit is configured to:
determine, using the one or more sensors, a first metric corresponding to a first state of a sympathetic nervous system of the patient;
control at least one of the balloon or the pump to alter a blood flow within renal artery, thereby changing the sympathetic nervous system from the first state to a second state;
determine, using the one or more sensors, a second metric corresponding to the second state of the sympathetic nervous system; and
provide, to a display in communication with the processor circuit, an output based on the first metric and the second metric.
